(19) United States
(12) Patent Application Publication
(10) Pub. No.: US 2023/0112573 A1
(43) Pub. Date: Apr. 13, 2023

(54) BIOMATERIALS AND RELATED METHODS AND KITS

(71) Applicant: OREGON HEALTH & SCIENCE UNIVERSITY, Portland, OR (US)

(72) Inventors: Luiz BERTASSONI, Portland, OR (US); Avathamsa ATHIRASALA, Portland, OR (US)

(21) Appl. No.: 17/905,147

(22) PCT Filed: Feb. 26, 2021
(86) PCT No.: PCT/US2021/020014

§ 371 Date: Aug. 26, 2022

Related U.S. Application Data
(60) Provisional application No. 62/983,482, filed on Feb. 28, 2020.

(51) Int. Cl. A61L 27/36; A61L 27/16; A61L 27/38; A61L 27/52; B33Y 70/00

(52) U.S. Cl. CPC A61L 27/3608; A61L 27/3633; A61L 27/16; A61L 27/3808; A61L 27/3834; A61L 27/3625; A61L 27/52; A61L 27/3687; A61L 27/3691; B33Y 70/00; A61L 2300/802; A61L 2430/40

(57) ABSTRACT

Biomaterials for tissue regeneration and engineering applications and methods of making and use thereof are described, as well as constructs and kits derived from the biomaterials. The biomaterials can be derived from extracellular matrix and functionalized to make them crosslinkable and amenable to tuning of their material properties.

### RELATED APPLICATIONS

This application claims priority to U.S. Provisional Patent Application No. 62/983,482 filed on Feb. 28, 2020, entitled “BIOMATERIALS AND RELATED METHODS AND KITS,” which is hereby incorporated herein by reference in its entirety.

### FEDERALLY SPONSORED RESEARCH OR DEVELOPMENT

This invention was made with government support under Grant Nos. R01DE026170 and 3R01DE026170-03S1 awarded by the National Institute for Dental and Craniofacial Research (National Institutes of Health). The government has certain rights in the invention.

### TECHNICAL FIELD

The present disclosure relates to biomaterials and methods for use in tissue regeneration applications.

### BACKGROUND

The rational design of biomaterials for regenerative applications has long sought to approximate the biological composition of the native extracellular matrix (ECM). The main components of the human ECM are collagens, proteoglycans, laminin, fibronectin, and elastin, which, along with other matrix macromolecules and growth factors, link together to form a structurally stable network that contributes to the mechanical properties of different tissues. The ECM, however, is tissue-specific, where cells secrete matrix molecules based on their local conditions, such as biological function, mechanical loading, hypoxia, and variability in nutrient concentration. Furthermore, the composition of the ECM varies dynamically through life to regulate various processes of development, differentiation, and remodeling. The bioactive nature of the native ECM of various tissues has been well established. Therefore, researchers have capitalized on the cell instructive and regenerative potential of these naturally bioactive materials to develop matrix-derived scaffolds for regenerative applications, examples of which include repair of bone, cartilage, brain and spinal cord, pancreas and a host of other tissues.

Despite the remarkable capacity of ECM-derived proteins to facilitate tissue regeneration and healing and their inherent capacity to self-assemble into structured hydrogels, their primary disadvantage remains the lack of control over their material properties, such as stiffness, degradability, microporosity, etc; all of which have been shown to significantly influence cell behavior. Additionally, their weak polymerization chemistry and resultant material properties do not usually support the fabrication of 3D constructs with the complex microarchitectural features that are typically associated with native tissues.

For example, autologous bone grafting has a long history as the gold standard in bone repair and regeneration, due to its high osteoconductivity, osteogenicity, and osteoinductivity. However, the limitations associated with this approach (e.g. potential scarcity, impact on the patient of harvesting the bone) have spurred the continued search for xenogenic materials, such as allografts and synthetic materials, that may be effective alternatives for promoting regeneration. One such material is demineralized bone matrix (DBM), which is an established tool for bone repair in the clinical setting. As native collagen is the principal component of the bone ECM, demineralized bone matrix materials share the drawback of a lack of control over mechanical properties. From an engineering point of view, this is a constraint in regulating tissue mechanics, which is an important factor in determining cell behavior and differentiation.

### BRIEF DESCRIPTION OF THE DRAWINGS

FIG. 1 Synthesis and chemistry of methacrylated bone extracellular matrix material (bECM or BoneMA). (A) Schematic depicting the synthesis of BoneMA wherein osteochondral/osteoarticular bone were powdered, demineralized, and processed for removal of lipids and decellularization procedures to extract bone matrix proteins. The processed bone matrix was then modified by methacrylation to form BoneMA macromers (B) through i) an addition reaction with methacrylic anhydride where methacrylate groups are linked to the pendant amine groups. ii) BoneMA is polymerized by visible light in the presence of LAP photoinitiator through crosslinking of these methacrylate groups to form BoneMA hydrogel. Comparison of the NMR spectra of (C) unmodified bone matrix and (D) BoneMA showed a peak occurring between 5.3 and 6.3 ppm representing the vinyl group of methacrylates in BoneMA, in addition to the proton signal for aromatic amino acid and lysine groups in the bone matrix, thereby confirming derivatization of bone matrix to form BoneMA.

FIG. 2 Physical characterization of BoneMA. The mechanical properties of the BoneMA were tunable by light exposure with (A) the elastic modulus increasing gradually as a function of crosslinking time from 15 to 30 and 45 sec (*p<0.05; ***p<0.001). Similarly, the apparent pore size, as visualized by SEM images of (B) freeze-dried (Scale—100 μm) and (C) critical point dried BoneMA samples (Scale—2 μm), decreased with crosslinking duration from (i) 15 sec to (ii) 30 and (iii) 45 sec.

FIG. 3 Shear storage modulus of BoneMA and GelMA. A) The shear storage modulus of BoneMA increased during the first 100 seconds of crosslinking. During this period, the shear storage modulus reached approximately 500 Pa, and beyond 100 seconds, the value reached approximately 750 Pa, where the curve remained a constant after 150 seconds. B) GelMA had a sharp increase in storage modulus during the first 50 seconds, and the value reached approximately 2 kPa. The curve hit a constant at 125 seconds when the shear storage modulus reached a value of approximately 2.5 kPa.

FIG. 4 Injectability of BoneMA microgels. (A-F) Image sequence of BoneMA microgel delivery through a gauge 18 needle. The microgels were easily injected through the syringe and remains confined within the mold.

FIG. 5. Physical characterization of GelMA. (A) The elastic modulus of GelMA hydrogels increased as a function of crosslinking duration starting from 1.5 kPa at 15 sec to 3 kPa and 4.3 kPa at 30 and 45 sec, respectively. Meanwhile, apparent pore size as evidenced by SEM images of (B) freeze dried and (C) critical point dried GelMA samples showed a decrease in pore size from (i) GelMA hydrogels crosslinked for 15 sec to those that were crosslinked for (ii) 30 and (iii) 45 sec. (Scale—5 μm). (*p<0.05; **p<0.005; ***p<0.001).

FIG. 6A) Apparent diffusion of rhodamine dye, as measured by its fluorescence, was higher in BoneMA hydrogels polymerized for 15 sec in comparison with those that were polymerized for 30 and 45 sec, suggesting that the less crosslinked hydrogels were more permeable. (B) For the swelling analysis, BoneMA hydrogel discs (n=6) were stored in DPBS at 37 C for a day, blot dried, and the wet swollen weight was recorded. After weighing, the samples were lyophilized, and the dried samples were weighed again to record their corresponding dry weight. The equilibrium swelling ratio was calculated as the ratio of wet mass to the dry mass of the hydrogel. The swelling properties of all the samples were identical over the given time period. Scale bar—50 μm.

FIG. 7 Cytocompatibility of BoneMA. (A) Representative images of HDPSCs stained for live (blue) and dead (green) cells on days 1, 3, and 7 after encapsulation in BoneMA hydrogels crosslinked for 15, 30 and 45 sec showed (B) very high cell viability across all groups and time points.

FIG. 8 Cytocompatibility of GelMA hydrogels. Representative images of hDPSCs encapsulated in GelMA hydrogels crosslinked for 15, 30, and 45 secs and stained for live (blue) and dead (green) cells at 1, 3 and 7 day time points showed a high degree of viability across all conditions and time points. Scale bar—400 μm.

FIG. 9 Vascular network formation in BoneMA and GelMA. Representative fluorescence images of GFP-HUVECs in (A) BoneMA and (B) GelMA hydrogels, each crosslinked for 15, 30, and 45 sec showed better vascular network formation in the less crosslinked hydrogels. Importantly, capillary formation started earlier and grew faster in BoneMA hydrogels in comparison to that in GelMA hydrogels crosslinked similarly. (Scale bar—400 μm).

FIG. 10 BoneMA hydrogels (with an elastic modulus of 1.56 0.1 kPa) were more vasculogenic than GelMA hydrogels of similar stiffness (elastic modulus 1.57 0.3 kPa) with visibly better vascular network formation by HUVECs in BoneMA within one day of encapsulation. (Scale bar—400 μm).

FIG. 11 Characterization of vascular networks in BoneMA and GelMA after 3 days. Quantitative analysis of the vessel networks measured the (A) segments, (B) end points, (C) junctions, and (D) vessel length. The total vessel length after 3 days was significantly higher in BoneMA15 than GelMA15 (p<0.005), while there was no statistically significant difference among BoneMA30 and GelMA30, and BoneMA45 and GelMA45. (*p<0.05; **p<0.005; ***p<0.001; ****p<0.0001).

FIG. 12 Representative fluorescence microscopy image demonstrating the range of patterns and geometries achieved by printing HMSO-laden BoneMA with a DLP printer (Ember). The cells were stained with F-Actin (green) after printing to enable confirmation of the shape and structure of the printed constructs through fluorescence microscopy. (Scale—750 μm).

FIG. 13 Both positive and negative features ranging from 500-1000 μm in width were printed using BoneMA after a crosslinking time of 45 seconds. The resolution for the negative was better than positive for the 500 μm square print.

### DETAILED DESCRIPTION

In order to address the limitations of current materials used in tissue regeneration and engineering, a bone-derived biomaterial has been developed that comprises bone ECM proteins functionalized with (meth)acrylates, which render the biomaterial crosslinkable in the presence of a crosslinking agent while maintaining the biological advantages associated with the composition of the native ECM.

In an embodiment, a biomaterial having a tunable material property comprises a demineralized, decellularized bone extracellular matrix material (bECM) functionalized with a crosslinkable (meth)acrylate monomer. As used herein, “(meth)acrylate” encompasses acrylates and methacrylates as well as alkyl esters thereof, i.e., alkyl acrylates and alkyl methacrylates. As the bECM is derived from bone extracellular matrix, in a particular aspect the bECM can comprise elements of the organic component of bone extracellular matrix, including but not limited to collagen, non-collagenous proteins, and proteoglycans. In a particular embodiment, one or more side chains of one or more of the proteinaceous components in the bECM are functionalized with the crosslinkable (meth)acrylate monomer. In accordance with the present disclosure, the biomaterial comprises bone extracellular matrix material that is demineralized and decellularized. Therefore, in an aspect inorganic components of bone extracellular matrix, including but not limited to hydroxyapatite and other salts of calcium and/or phosphate, are substantially absent from the biomaterial or are present in very small amounts. In another aspect, cells and cellular material that were native to the bone extracellular matrix from which the bECM is derived are also substantially absent from the biomaterial or are present in very small amounts.

In accordance with the present disclosure, protein (meth)acrylation is employed to endow the matrix with crosslinkable moieties that allow for the control of the final mechanical properties of the biomaterial. In a particular embodiment, the biomaterial further comprises a crosslinking agent. It will be recognized by those of skill in the art with the aid of the present disclosure that a number of approaches and agents may be suitably employed to initiate crosslinking in the biomaterial. Crosslinking initiators include, but are not limited to, light, pH, temperature, hydration, and various chemical agents. Initiators that arise from the physical or chemical environment of the material to be crosslinked (e.g. pH or temperature) can allow for self-assembly of the crosslinked material.

It is contemplated that photopolymerization of bone extracellular matrix proteins using light sensitive moieties enables formation of hydrogel scaffolds with precisely tuned and reproducible microscale architectures and physico-mechanical properties, through modulation of light exposure. Furthermore, the photocrosslinkable nature of this material allows for straightforward bioprinting of cell-laden micro-tissue constructs using a digital light processing (DLP) based approach, which is advantageous over conventional methods of fabrication of demineralized and de-cellularized bone. Accordingly, in an embodiment, the crosslinking agent is a photoinitiator. In a specific embodiment, the photoinitiator is selected from the group consisting of lithium phenyl-2,4,6-trimethylbenzoyl phosphinate, lithium acylphosphinate, 2-hydroxy-1-(4-(hydroxyethoxy)phenyl)-2-methyl-1-propanone2-Benzyl-2-(dimethylamino)-4′-morpholinobutyrophenone, 4′-tert-Butyl-2′,6′-dimethylacetophenone, 2,2-Diethoxyacetophenone, 2,2-Dimethoxy-2-phenylacetophenone, a blend of Diphenyl(2,4,6-trimethylbenzoyl)phosphine oxide and 2-hydroxy-2-methylpropiophenone, 4′-Ethoxyacetophenone, 3′-Hydroxyacetophenone, 4′-Hydroxyacetophenone, 1-Hydroxycyclohexyl phenyl ketone, 2-Hydroxy-4′-(2-hydroxyethoxy)-2-methylpropiophenone, 2-Hydroxy-2-methylpropiophenone, 2-Methyl-4′-(methylthio)-2-morpholinopropiophenone, 4′-Phenoxyacetophenone, or combinations thereof. As will be recognized by those of skill in the art with the aid of the present disclosure, the amount of photoinitiator or other crosslinking agent included in the biomaterial can be selected based on the identity of the crosslinking agent and the desired results. In a more specific embodiment, the photoinitiator is lithium phenyl-2,4,6-trimethylbenzoyl phosphinate and is present in an amount of from about 0.05% w/v to about 5% w/v. As given photoinitiator may be activated by light having wavelengths within a particular range. In a particular embodiment, the biomaterial comprises a photoinitiator having an activation wavelength in the ultraviolet range. In another particular embodiment, the biomaterial comprises a photoinitiator having an activation wavelength in the visible range. In a specific exemplary embodiment, the photoinitiator has an activation wavelength of from about 200 nm to about 500 nm. In another specific exemplary embodiment, the photoinitiator has an activation wavelength of from about 400 nm to about 700 nm.

As the primary component of the biomaterial, the amount of bECM contained in the biomaterial can be selected according to the desired material properties or to be suited for particular uses. In an embodiment, the bECM is present in an amount of from about 0.5% w/v to about 40% w/v. The biomaterial can also include biological material that promotes biological processes involved in tissue regeneration, such as vasculogenesis or the invasion of appropriate progenitor cell types. Accordingly, in an embodiment, the biomaterial comprises a population of living cells that are not autologous to the ECM from which the biomaterial is derived. In specific embodiments, the population can comprise vascular endothelial cells and/or stem cells. In another embodiment, the biomaterial includes microvascular fragments. In addition, the components of the biomaterial can be combined in a carrier that is suitable for the intended use. In a particular embodiment, the carrier is a pharmaceutically acceptable medium. Nonlimiting examples include bioinert solvents such as saline and water.

Initiation of crosslinking in the biomaterial results in formation of a hydrogel having properties that result at least in part from the particular composition of the biomaterial, the degree of crosslinking, the crosslinking agent, and the parameters of crosslinking initiation. Accordingly, a hydrogel construct can comprise a demineralized, decellularized bone extracellular matrix material (bECM) functionalized with a crosslinkable (meth)acrylate monomer, wherein the bECM is at least partially crosslinked. As one use of such a construct is to promote tissue regeneration in the site where it is placed, the hydrogel construct can include biological materials selected to promote regeneration. In this way the construct can serve as a device for delivering such material to a tissue to be regenerated. Accordingly, in an embodiment, the hydrogel construct comprises a population of living cells that are not autologous to the ECM from which the biomaterial is derived. In specific embodiments, the population can comprise vascular endothelial cells and/or stem cells. In another embodiment, the hydrogel construct includes microvascular fragments. The hydrogel construct can be formed in situ, e.g., uncrosslinked biomaterial can first be delivered to a tissue site to be regenerated, after which crosslinking is initiated. In other applications the hydrogel construct can be formed before delivery to the site. Accordingly, in embodiments the hydrogel construct can be processed to facilitate storage, handling, and delivery. In a specific embodiment, the hydrogel construct is lyophilized.

An aspect of the embodiments described herein is that the composition of the crosslinkable biomaterial provides for tunability of material properties of the resultant hydrogel. These material properties include stiffness, microporosity, degradability, and other properties that can influence the behavior of cells involved in regenerating a particular tissue. The biomaterial of the present disclosure provides a number of avenues for tuning these mechanical properties, including changing the (meth)acrylate concentration, crosslinker concentration, curing time and/or combination of these parameters, which is advantageous in controlling the stem cell microenvironment. For photocrosslinkable biomaterial in particular, in situ photopolymerization is possible using light sources that are used clinically, such as UV or visible light sources. It should also be noted that the present biomaterial provides a multi-component hydrogel that carries the proteolytic by-products of pepsinized collagen and resulting peptides of a wide range of non-collagenous proteins. This is in contrast to single component hydrogels, such as methacrylated gelatin (GelMA), methacrylated hyaluronic acid (meHA) or methacrylated tropoelastin (MeTro), which have only one major component like gelatin, HA, and tropoelastin, respectively. While not being bound to a particular theory, it is surmised that differences in physical properties and tunability thereof between biomaterials of the present disclosure and other hydrogel materials may be attributed to the heterogeneity in the molecular network that is formed in the cross-linked bone peptides (of different lengths and types) versus the more homogenous and controlled one-component matrices of other hydrogels.

With the foregoing in mind, in an embodiment, a method of making a hydrogel construct, comprises introducing an amount of a biomaterial into a shaping device, and curing the biomaterial to form a hydrogel having a material property, where the biomaterial comprises a demineralized, decellularized bone extracellular matrix material (bECM) functionalized with a crosslinkable (meth)acrylate monomer; and a crosslinking agent. In a particular embodiment, the crosslinking agent is a photoinitiator, and the curing step comprises exposing the biomaterial to light for an exposure time so as to achieve the material property. Without being bound to a particular theory, it is surmised that the ability of a hydrogel scaffold to promote certain regenerative processes, e.g., microvascular network formation, is linked to the stiffness of the hydrogel. Accordingly, in a specific embodiment, the material property is an elastic modulus. It should be noted however, that other aspects of the biomaterial of the present disclosure may also contribute to enhanced regenerative processes. For example, the biomaterial can include pro-angiogenic peptides that are found in bone extracellular matrix and that persist in the biomaterial when it is made.

In accordance with the method, the shaping device is any device, structure, or system that can be used to impart a particular shape or dimension to the hydrogel construct, either before or during curing. In one embodiment, the shaping device is a mold into which a quantity of the biomaterial is placed and then cured. In an alternative embodiment, the shaping device is a microfluidic channel. In another embodiment, the shaping device is a three-dimensional (3D) printing platform. As noted above, biomaterial of the present disclosure can be used as a bioink for DLP bioprinting, where photocrosslinkability of the biomaterial is a key element. One of the highlights of the printed structures is the printability of the biomaterial in microdimensions leading to the formation of particular hydrogel constructs termed microgels. Microgels are a special class of materials that have gained attention in fabricating complex tissues. Microgels are crosslinked polymer networks in the micron range, which has considerable advantages over bulk hydrogels in the area of controlled release of drugs and protein, hydrolytic degradation, personalized medicine screening and microscale tissue engineering. In microscale tissue engineering, microgels are used as building blocks for the bottom-up building of hetero-architecture to mimic complex tissues. Accordingly, in a particular embodiment making a hydrogel construct comprises assembling a plurality of hydrogels to form such a structure.

Shape can be a significant factor in microgel geometry to create centimeter-scale tissue-like structures. For example, tightly packed cell-laden tissue-like structures can be created through bottom-up self-assembly in a multiphase environment (liquid-air system). Hexagonal cell-laden microgels form tissue-like structures through an interface-directed assembly process, while more complex building blocks like lock-and-key microgels allowed greater control over self-assembly. Since cell-laden biomaterial of the present disclosure allows printing in multiple complex shapes, similar lock-and-key shaped cell-laden microgels can be printed to create complex shapes and tissues. Also, culturing the hydrogels described herein with two or more cells can further address the complexity of building tissues. Therefore, these hydrogel constructs can be an effective ECM-based building block for bottom-up manufacturing of complex tissues. Injectability of microgels is another parameter that plays a significant role in therapeutic delivery for treating site-specific tissue damage like myocardial infarction, enhancing neovascularization and cellular differentiation. The injectability of the biomaterial of the present disclosure opens up venues for investigations in basic biology and clinically-oriented minimally invasive implantation procedures.

In an embodiment, a kit for use in tissue engineering comprises a biomaterial in a container, where the biomaterial comprises bECM functionalized with a crosslinkable (meth)acrylate monomer and a crosslinking agent. In a more specific embodiment, the the crosslinking agent is a photoinitiator. In a particular embodiment, the kit further comprises instructions for delivering an amount of the biomaterial and curing the bECM to form a hydrogel. In other specific embodiments, the kit further comprises a delivery device for delivering an amount of the biomaterial and said delivery device can specifically be configured to be operably connected to the container for delivering the biomaterial directly from the container. In other particular embodiments, the biomaterial is formulated as a paste or alternatively as a lyophilized powder.

In GelMA synthesis, pH is typically maintained at 7.4 by phosphate buffer, which introduces methacryloyl groups to the reactive amine and hydroxyl groups of the amino acid residues. The main limitation in this process is that the referred pH limits the reactivity of amine and hydroxyl groups. In accordance with the present disclosure, this problem is addressed by improved synthesis protocol, where a higher pH is maintained during the reaction using a buffer (e.g. carbonate bi-carbonate) to keep the isoelectric point (IEP) of the bone proteins high, keeping the free amino groups of lysine neutral and allowing for it to react with a (meth)acrylic reagent. Accordingly, in an embodiment, a method of making a biomaterial having a tunable material property, comprises the steps of demineralizing powdered bone by treating the powdered bone with acid to produce demineralized bone material; extracting lipids from the demineralized bone material; decellularizing the demineralized bone material to produce demineralized bone extracellular matrix material; solubilizing collagen in the demineralized bone extracellular matrix material; and reacting the demineralized bone extracellular matrix material with a (meth)acrylic reagent to produce a crosslinkable bone extracellular matrix material, wherein said reacting is performed at a pH of from about 8 to about 10. In another specific embodiment, the reacting step is performed with an amount of (meth)acrylic reagent of from about 0.1 ml to about 0.3 ml per gram of demineralized bone extracellular matrix material. In another embodiment, the reacting step is performed at a reaction temperature of from about 30° C. to about 55° C. In still another embodiment, the reacting step is performed for a reaction time of from about 1 to about 5 hours.

In a specific embodiment, the (meth)acrylic reagent is methacrylic anhydride. However, it will be recognized by those of skill in the art with the aid of the present disclosure, that (meth)acrylate reagents having reactive (meth)acrylate groups can be used to functionalize the proteins of the bECM, including without limitation, salts and copolymers.

The inventors have also found that ECM protein yield from this process can be enhanced by centrifugation and filtering of intermediate products and by-products of certain steps. For example, in an embodiment, the method of making the biomaterial further comprises centrifuging a solution produced by any one of the above steps to produce a precipitate and a supernatant; filtering solids of a size from the supernatant; and then performing the subsequent step on the solids.

### EXAMPLE 1. PREPARATION OF DEMINERALIZED AND DECELLULARIZED BONE MATRIX

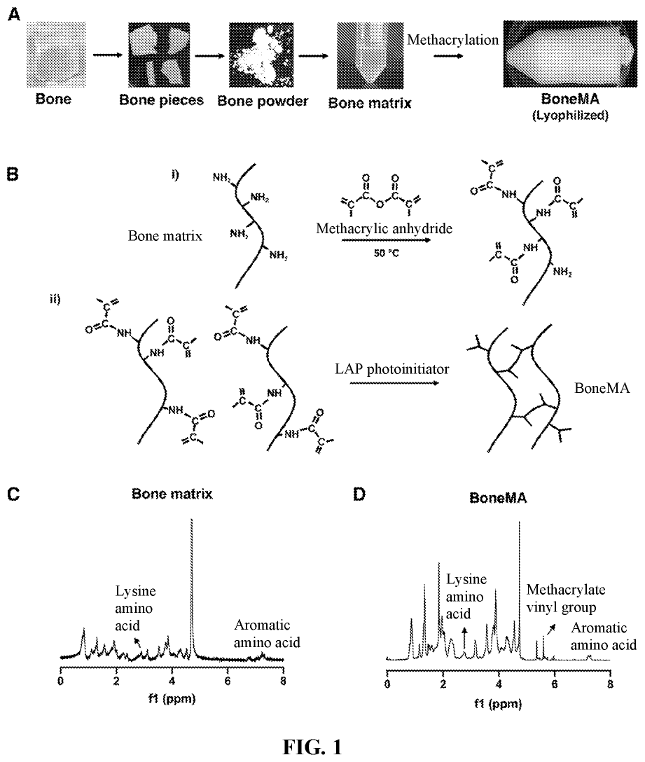

Demineralized and decellularized bone ECM bone was extracted as summarized in FIG. 1A. In short, fresh human osteochondral/osteoarticular bone fragments were scraped thoroughly to remove residual tissue and rinsed with Dulbecco's phosphate buffered saline (DPBS) solution containing penicillin (100 IU/ml) and streptomycin (100 μg/m1) (Corning, USA). The bone fragments were sectioned using a low-speed diamond saw (Struers Accutom-5) and the resulting sections were then ground to a fine powder using a cryogenic freezer/mill (SPEX 6700, USA). The finely ground bone powder was demineralized under agitation using 0.5 N HCl (25 ml per gram) for 24 hours and the insoluble portion of the bone matrix was retrieved by vacuum-filtration after rinsing with distilled water. After lipid removal by solvent extraction with 1:1 (v/v) chloroform (Sigma-Aldrich, USA)-methanol (Acros Organics, USA) solution for 1 hour, followed by rinsing with methanol and distilled water, the demineralized bone matrix was flash-frozen in liquid nitrogen and lyophilized overnight (Labconco, USA). Subsequently, the demineralized bone matrix was decellularized with 0.05% trypsin-0.02% ethylenediaminetetraacetic acid (EDTA) at 37° C., 5% CO₂ under continuous agitation for 24 hours and then rinsed with DPBS containing penicillin (100 IU/ml) and streptomycin (100 μg/m1). Insoluble proteins were retrieved after each step by centrifuging the solution at 1500 rpm for 30 mins and decanting the solution to collect the precipitates. The decanted solution was vacuum filtered using a pore size of 200 microns to recover additional proteins.

Following this, the processed bone matrix was subjected to enzymatic digestion by pepsin (1 mg/ml in 0.01 N HCl), where a suspension of 10 mg of matrix per ml of pepsin was stirred under agitation for 96 hours until the matrix was solubilized. Finally, the solubilized matrix was centrifuged for 30 min at 4° C., filtered under vacuum and stored at −20° C.

### EXAMPLE 2. SYNTHESIS OF METHACRYLATED BECM

For the synthesis of methacrylated bECM (also referred hereinafter in both its uncrosslinked and crosslinked forms, as applicable, as “BoneMA”), the frozen demineralized and decellularized bone matrix was lyophilized for two days. Next, 10% (w/v) of the lyophilized matrix was dissolved in 0.25M carbonate-bicarbonate buffer, and the pH was adjusted to 9 using 4M NaOH or 6M HCl under constant stirring at 50° C. Once the bone matrix was dissolved, methacrylic anhydride (0.2 ml/g of bone matrix) was added to the solution in a dropwise manner under constant stirring while maintaining the temperature. The reaction was allowed to proceed for 3 hours, and the pH was maintained at 9 by adding 4M NaOH. After the completion of the reaction, the modified bone matrix solution was diluted 4× times with warm deionized water. Unreacted methacrylic anhydride was removed from the solution by dialyzing it against deionized water for 24 hours, following which the solution was filtered, lyophilized and stored at −80 ° C. until further use (see FIGS. 1A, B). In order to confirm the methacrylation of the bone matrix, we performed nuclear magnetic resonance (NMR) on the material using a Bruker 400 MHz Avance II+ spectrometer with a magnetic field strength of 400 MHz and chloroform-d as the solvent. The NMR spectra readout of the reaction product was compared with that of unmodified bone ECM as a control.

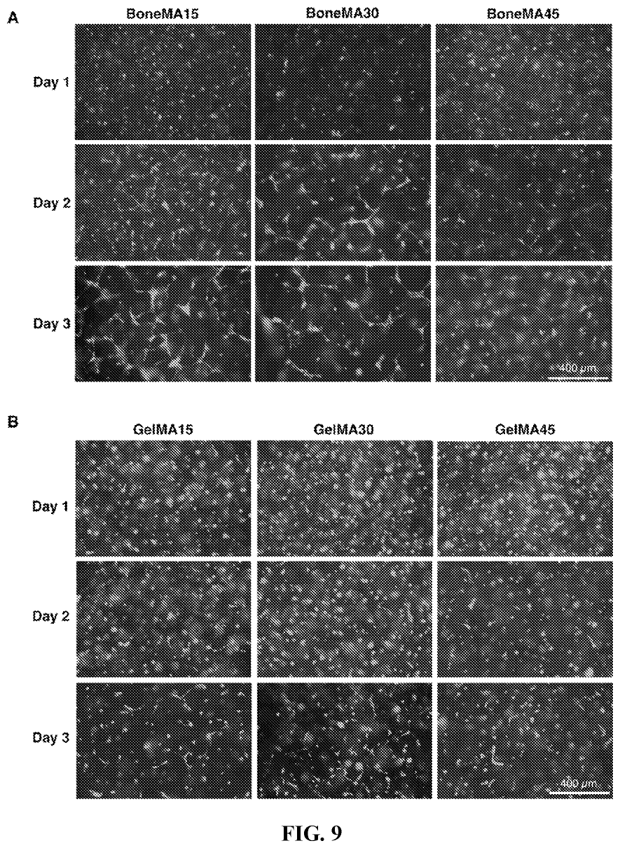

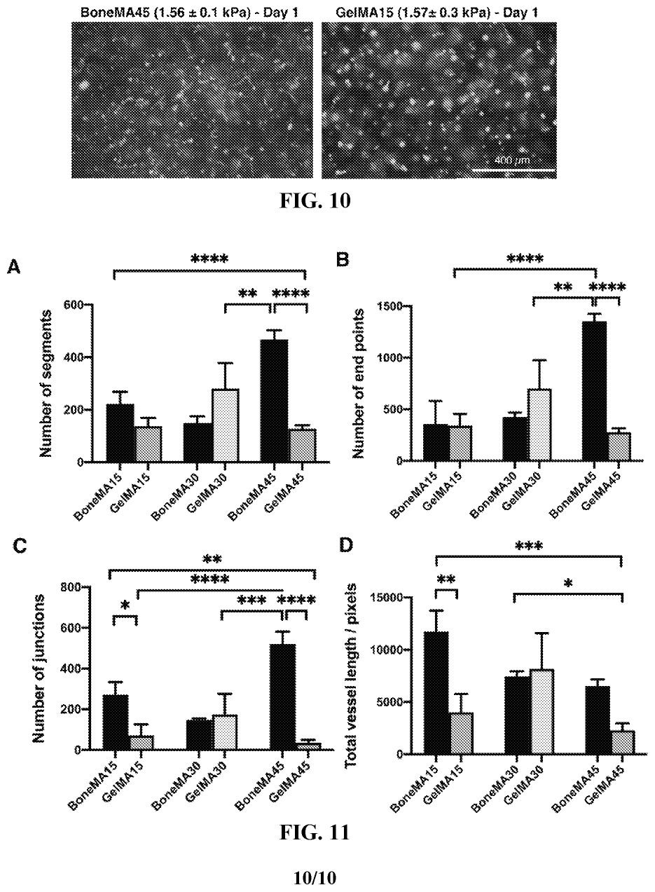

The spectra of unmodified bone matrix showed the expected peaks corresponding to the amino acids containing primary amines and aromatic groups (FIG. 10), whereas the bECM macromer had peaks between 5 and 6 ppm, indicative of the presence of the vinyl component of methacrylates (FIG. 1D), thus confirming functionalization of the bone matrix proteins with methacrylate.

### EXAMPLE 3. PREPARATION OF GELATIN METHACRYLOYL (GEIMA)

Gelatin methacryloyl (GelMA) was used as a control to compare against the biological properties of BoneMA. GelMA was synthesized as per the protocol described by Nichol et al. Porcine skin type A gelatin (10% w/v) (Sigma, St Louis, Mo., USA) was dissolved in Dulbecco's phosphate buffered saline (DPBS, Sigma) warmed to 50° C. to which, 8% (v/v) methacrylic anhydride (Sigma) was added dropwise and allowed to react for 2 h. Next, the solution was diluted 5× times with DPBS and dialyzed with 12-14 kDa dialysis tubing against warm distilled water (45±5° C.) for 5 days. The warm water was changed two times a day for 5 days. The resulting methacrylated prepolymer was lyophilized and stored at room temperature until further use. For GelMA sample synthesis, GelMA was crosslinked for 15 sec, 30 sec, and 45 sec using the bioprinter as described previously, and the resultant constructs are identified as GelMA15, GelMA30, and GelMA45 respectively. The GelMA samples were treated similarly to BoneMA for SEM and live/dead analysis

### EXAMPLE 4. HYDROGEL PREPARATION

The freeze-dried methacrylated bECM was dissolved in DPBS (5% (w/v)) with 0.15 w/v% Lithium phenyl-2,4,6-trimethylbenzoylphosphinate (LAP, Tokyo Chemical Industry, L0290) photoinitiator. A digital light processing (DLP) 3D printer (Ember, Autodesk) was used to prepare hydrogel samples for different experiments by exposing the hydrogel prepolymer to 20 mW/cm² of light (405±5 nm) for 15, 30, and 45 sec. For physical and mechanical characterization, samples were printed with 5 mm in diameter and 1.5 mm in thickness, whereas biological characterization tests used 450 thick specimens printed with geometrical shapes described below (N=5).

### EXAMPLE 5. ANALYSIS OF MECHANICAL PROPERTIES

The hydrogel mechanical properties were measured using a DHR-1 rheometer (TA Instruments) fitted with a UV curing accessory containing a bandpass filter for 405±5 nm. The intensity of the light from an Acticure® 4000 (EXFO) coupled with the rheometer was matched to the DLP printer at 20 mW/cm² using a power meter (Power Max5200, Molectron). Shear modulus measurements were performed as a function of crosslinking time under 0.1% strain and frequency of 10 Hz and were converted to elastic modulus using a previously reported equation.

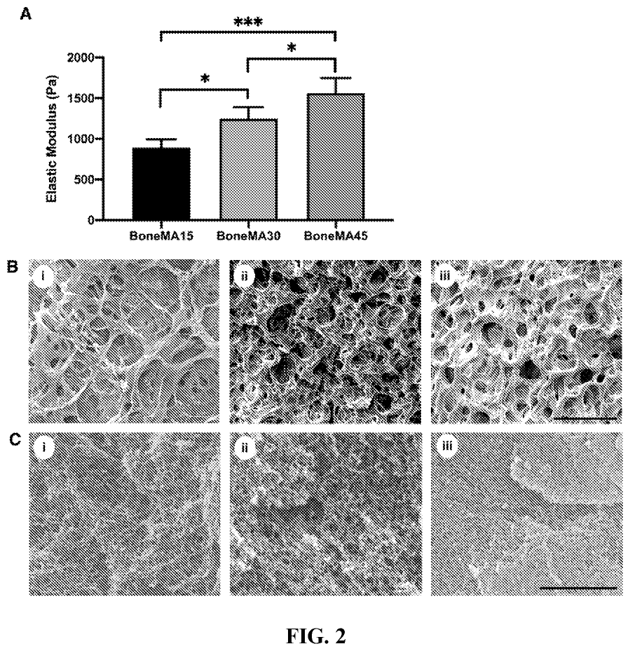

The tunability of the mechanical properties of the newly formed methacrylated material was examined as a function of light exposure by comparing the elastic moduli of hydrogels polymerized for 15, 30 and 45 seconds, using the DLP 3D printer as a light source. The elastic modulus of the material was found to increase significantly as a function of the duration of light exposure, starting at 0.9 kPa when crosslinked for 15 s, and increasing gradually to 1.3 (p<0.05) and 1.5 (p<0.001), at 30 and 45 s respectively (FIG. 2A).

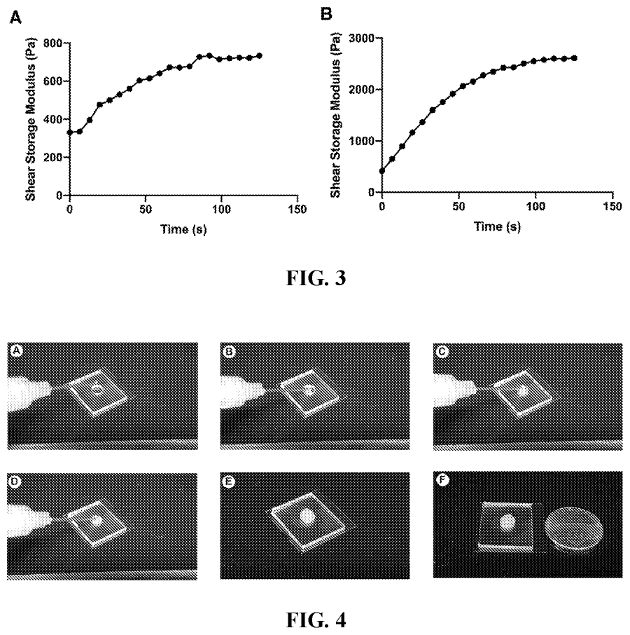

The rheological studies revealed relevant information regarding the photocrosslinking process of the methacrylated bECM (FIG. 3A). The gradual slope until 125 seconds indicates that the modulus increased with an increase in photocrosslinking time, as expected. GelMA, too showed a gradual slope in shear storage modulus, but it was about 2.6 kPa at 125 seconds (FIG. 3B), which was substantially higher than BoneMA. This indicates that BoneMA is a much softer material than GelMA, which makes for a desirable material when lower stiffness is needed, such as in vascular regeneration. With respect to bioprinting, specifically, the storage modulus of the material also plays an important role. It is reported that when a hydrogel has a storage modulus in the order of 100-1000 Pa, it is highly suitable for bioprinting, especially for extrusion. Therefore, the shear modulus of BoneMA may also allow it to be used as a bioink for extrusion purposes. Also, the injectability of BoneMA microgels (FIG. 4) opens up venues for investigations in basic biology and clinically-oriented minimally invasive implantation procedures

### EXAMPLE 6. SCANNING ELECTRON MICROSCOPY (SEM) IMAGING

The hydrogel samples were prepared according to the above procedure for all time points. For SEM, the BoneMA hydrogel was immersed in 5 mL DPBS at 37° C. for 24 h (n=5). Next, samples were fixed with 2.5% glutaraldehyde solution for 1 hour. After fixing, the samples were dehydrated for 10 minutes in a series of ethanol solutions of concentration 25%, 50%, 75%, 90%, and 100%, respectively. After dehydration, samples were critical point dried for 3 hours. These dried samples were coated with gold/palladium and imaged with FEI Quanta 200 SEM at 15 kV.

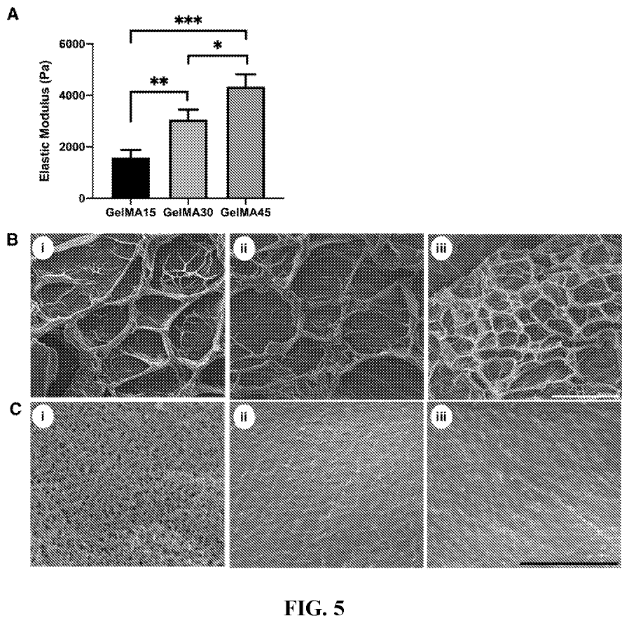

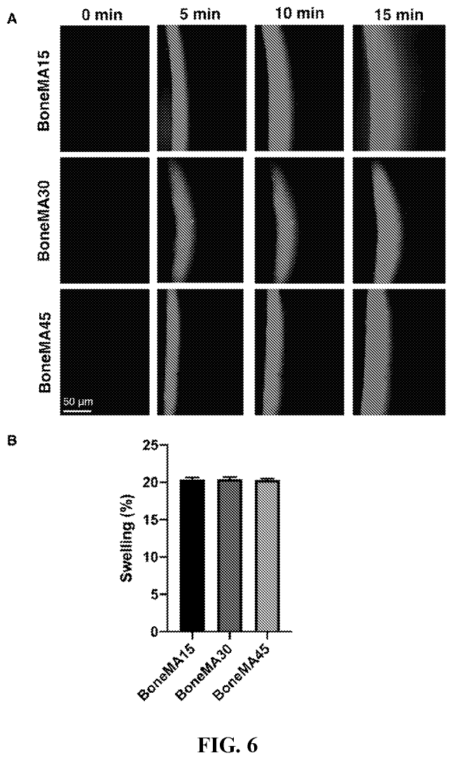

SEM images showed the presence of the typical pore-like microstructures that are commonly observed in covalently crosslinked hydrogels with BoneMA crosslinked for 15 seconds having distinctly larger pores than its more crosslinked counterparts (FIG. 2B). Critical point drying of the samples enabled imaging of the highly interconnected fibrous micromorphology of the hydrogels (FIG. 2C), suggesting that, despite the methacrylation of the solubilized and pepsinized fibrillar proteins in the bone matrix, a semi-fibrillar hydrogel still formed on the nanoscale. Notably, the same porous and fibrous morphology was observed in GelMA hydrogels (FIG. 5B, C). However, while there was no significant difference in the swelling mass ratio of BoneMA as a function of crosslinking time, dye permeability studies showed that the rate of diffusion through each of these BoneMA hydrogels mirrored their apparent pore size (FIG. 6).

### EXAMPLE 7. CELL VIABILITY

Since different aspects and applications of BoneMA were being tested, samples were prepared using different cell lineages for various experiments. To that end, human dental pulp stem cells (HDPSC) and human mesenchymal stem cells (HMSC) were used to assess the general bioink properties, such as cytocompatibility and bioprintability, while green fluorescent protein (GFP)-expressing human umbilical vein endothelial cells (HUVECs) (cAP0001GFP, Angio-proteomie, USA) were employed to investigate the vasculogenic potential of the synthesized biomaterial. HDPSCs (Lonza, USA) and HMSCs were each cultured in α-MEM medium supplemented with 10% (v/v) fetal bovine serum and 1% (v/v) penicillin-streptomycin. HUVECs were cultured in Endothelial Cell culture medium (Vasculife-VEGF, Lifeline Cell Technologies) on 0.1% gelatin-coated substrates. Cells were maintained in an incubator at 37° C., 5% CO₂ incubator, and the medium was replaced every 2 days.

In order to assess the cytocompatibility of BoneMA, HDPSCs were trypsinized and resuspended in BoneMA (5% (w/v)) containing LAP (0.15% (w/v)) at a cell density of 5×10⁵ cells/mL. This cell-laden prepolymer solution was photopolymerized as described above for 15, 30, and 45 sec. Cell viability was measured on days 1, 3, and 7 post-encapsulation using a membrane permeability based fluorescent live/dead staining kit (Molecular Probes), and the fraction of live cells was estimated using ImageJ software from at least 3 distinct regions per sample (N=5).

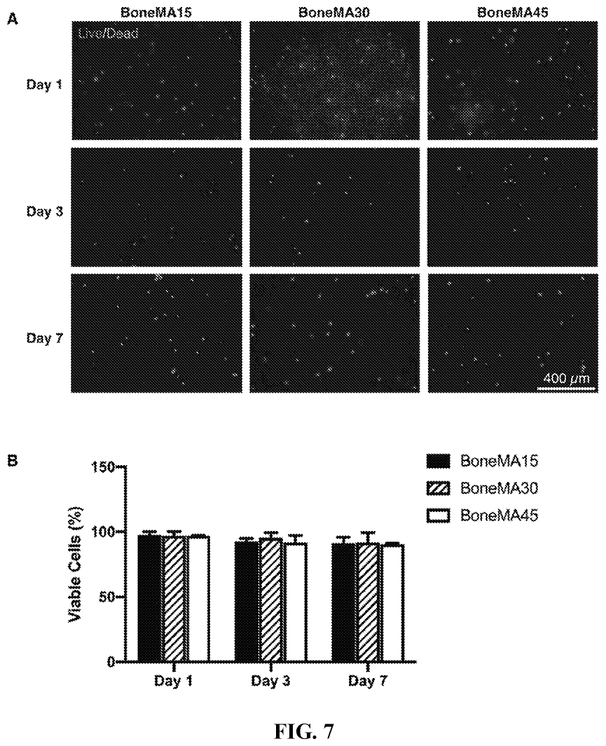

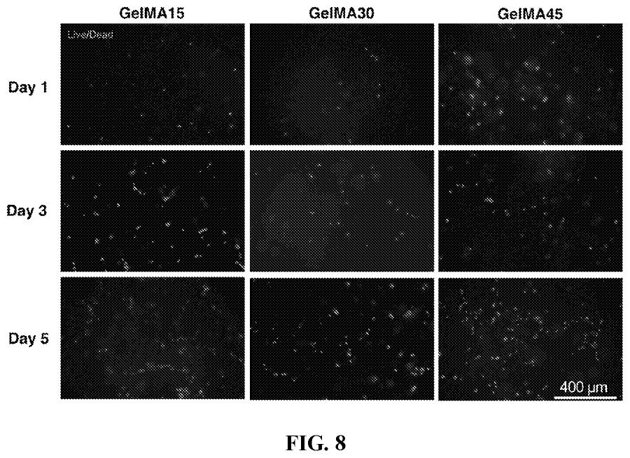

HDPSCs remained highly viable through at least 7 days in culture within all BoneMA hydrogel constructs, with average viability of 91, 94 and 90% for BoneMA crosslinked for 15, 30 and 45 seconds on day 7, respectively (FIGS. 7A, B). These results confirmed the cytocompatibility of the biomaterial in comparison with more established hydrogel scaffolds, such as GelMA (FIG. 8), which was used as a control.

### EXAMPLE 8. FABRICATION OF VASCULARIZED HYDROGELS

The vasculogenic potential of BoneMA was examined in comparison to GelMA, which was synthesized with a comparable degree of methacrylation and photo-crosslinked for the same amounts of time. To that end, HUVECs were encapsulated in BoneMA hydrogels (5% (w/v)) containing LAP (0.15% (w/v)) at a cell density of 1×10⁷ cells/ml, crosslinked for 15, 30 and 45 s as described previously. Vascular capillary network formation in these hydrogels was characterized daily for 7 days and quantified for the number of segments, number of end points and junctions, as well as the total length of the network using an ImageJ.

In order to assess the vasculogenic potential of BoneMA, we compared vascular network formation in HUVEC-laden hydrogels crosslinked for 15, 30, and 45 sec (FIG. 9A) against similarly crosslinked GelMA hydrogels (FIG. 9B), which was used as a control. Better vascular network formation was observed in the less crosslinked hydrogels at each time point in both BoneMA and GelMA suggesting that softer mechanics and higher porosity supported the formation of capillaries. However, while the network formation began in as little as 24 h in BoneMA hydrogels, with almost fully established capillary formation by day 2 in BoneMA hydrogels, network formation was much slower in GelMA, which only started around day 2 and was more established during day 3. Since chemoattraction for vascular network formation is innately dependent on the physico-mechanical properties of the hydrogel, especially the stiffness, we directly compared capillary formation in BoneMA and GelMA hydrogels of comparable elastic moduli. Interestingly, BoneMA crosslinked for 45 seconds to an average modulus of 1.56 kPa, showed faster network formation than and GelMA crosslinked for 15 seconds to a comparable modulus of 1.57 kPA (FIG. 10).

Quantification of vascular network formation (FIG. 11) showed that the number of vascular segments was, in general, higher in BoneMA samples compared to that in GelMA. In particular, there was a significant difference between BoneMA exposed to 45 seconds of photopolymerization, and GelMA exposed for 30 seconds, as the number of segments increased in BoneMA by a factor of 1.6 (p<0.001). Similarly, there was a significant difference in the number of segments between samples BoneMA15 versus GelMA45 (p<0.0001), and BoneMA45 versus GelMA45 (p<0.0001). The number of endpoints, which represents the number of sprouts found at the ends of the vessel as well as the tip in side-sprouting networks, was also higher in BoneMA, suggesting an active network formation in the hydrogel. We also found a significant difference among GelMA30 versus BoneMA45 (p<0.01), GelMA15 versus BoneMA45 (p<0.0001), and BoneMA45 versus GelMA45 (p<0.0001), where the number of junctions was higher in BoneMA than GelMA. The total vessel length was also generally higher in BoneMA hydrogels. For instance, the total vessel length for BoneMA15 increased by a factor of 5.1 (p<0.001) in comparison to GelMA45, and 2.9 (p<0.005) for GelMA15. All these quantitative data show a very active network forming process occurring in BoneMA when compared to GelMA.

### EXAMPLE 9. BIOPRINTING OF BONEMA GEOMETRIES

To bioprint BoneMA in various geometrical shapes, specific print patterns were designed using CAD (Fusion 360, AutoDesk) and converted into image slices with the accompanying 3D printing software (Print Studio, Autodesk). The print patterns were designed arbitrarily with star, square, triangle, and rhombus shapes, as well as flower, spiral, concentric circles, and the OHSU logo. Shapes were printed with length and width dimensions that ranged from 600 μm (square) to 2.5 mm (OHSU logo) with a thickness of 450 μm, under printing exposures of 25 sec. For bioprinting of the said geometries, the bioink was formulated by mixing HMSCs with the BoneMA (5% (w/v)) pre-polymer containing LAP (0.15% (w/v)) at a concentration of 5×10⁵ cells/ml. The HMSO-laden bioprinted BoneMA was fixed with 4% (v/v) paraformaldehyde and permeabilized using 0.1% (v/v) Triton X-100. Next, the samples were treated with 1.5% (w/v) bovine serum albumin (Sigma Aldrich) in DPBS for 1 h, followed by Image-iT FX signal enhancer (Invitrogen, Calif.) for 30 min, after which they were immunostained with ActinGreen™ 488 ReadyProbes™ (Invitrogen).

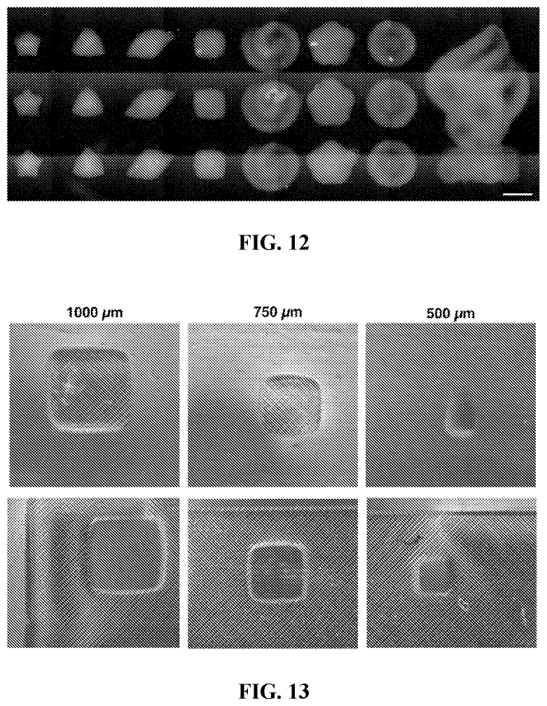

Fluorescence microscopy images of bioprinted patterns using BoneMA encapsulating HMSCs and immunostained for F-Actin (FIG. 12) show a variety of geometrical patterns of different sizes. Micropatterns that ranged from 600 μm (square) to 2.5 mm (OHSU logo) could be bioprinted in high throughput, where as many as 2.5 thousand microgels could be bioprinted in as little as 15 sec. Of note, these microgels can be bioprinted with the exact sample printing parameters optimized for vasculature formation, as shown in previous pictures, thereby allowing for fabrication of thousands of pre-vascularized microscaffolds at a time. We also printed the positive and negative features of BoneMA without cells with dimensions ranging from 500 μm to 1000 μm (FIG. 13). Importantly, these microgels can be injected through a standard syringe needle, thus forming a straightforward platform for the fabrication of pre-vascularized injectable microgels with advantageous vasculogenic properties.

All above data are presented as mean±standard deviation. Data were compared using one way ANOVA followed by Tukey posthoc test (α=0.05) with Graphpad Prism 8.

It will be apparent to those having skill in the art that many changes may be made to the details of the above-described embodiments without departing from the underlying principles of the invention. The scope of the present invention should, therefore, be determined only by the following claims.

1. A biomaterial having a tunable material property, comprising a demineralized, decellularized bone extracellular matrix material (bECM) functionalized with a crosslinkable (meth)acrylate monomer.

2. The biomaterial of claim 1, further comprising a crosslinking agent.

3. The biomaterial of claim 2, wherein the crosslinking agent is a photoinitiator.

4. The biomaterial of claim 3, wherein the photoinitiator is selected from the group consisting of lithium phenyl-2,4,6-trimethylbenzoyl phosphinate, lithium acylphosphinate, 2-hydroxy-1-(4-(hydroxyethoxy)phenyl)-2-methyl-1-propanone.

5. The biomaterial of claim 3, wherein the photoinitiator is lithium phenyl-2,4,6-trimethylbenzoyl phosphinate and is present in an amount of from about 0.05% w/v to about 5% w/v.

6. The biomaterial of claim 3, wherein the photoinitiator has an activation wavelength of from about 200 nm to about 500 nm.

7. The biomaterial of claim 3, wherein the photoinitiator has an activation wavelength of from about 400 nm to about 700 nm.

8. The biomaterial of claim 1, wherein the bECM is present in an amount of from about 0.5% w/v to about 40% w/v.

9. The biomaterial of claim 1, further comprising a population of living cells.

10. The biomaterial of claim 9, wherein the population of living cells includes one or more vascular endothelial cells.

11. The biomaterial of claim 9, wherein the population of living cells includes one or more stem cells.

12. The biomaterial of claim 1, further comprising one or more microvascular fragments.

13. The biomaterial of claim 1, wherein the BoneMA is present in a pharmaceutically acceptable medium.

14. A hydrogel construct comprising a demineralized, decellularized bone extracellular matrix material (bECM) functionalized with a crosslinkable (meth)acrylate monomer, wherein the bECM is at least partially crosslinked.

15. The hydrogel construct of claim 14, further comprising a population of living cells.

16. The hydrogel construct of claim 15, wherein the population of living cells includes one or more vascular endothelial cells.

17. The hydrogel construct of claim 15, wherein the population of living cells includes one or more stem cells.

18. The hydrogel construct of claim 14, further comprising one or more microvascular fragments.

19. The hydrogel construct of claim 14, wherein the hydrogel construct is lyophilized.

20. A method of making a hydrogel construct, comprising the steps:
a. introducing an amount of a biomaterial into a shaping device, wherein the biomaterial comprises:
i. a demineralized, decellularized bone extracellular matrix material (bECM) functionalized with a crosslinkable (meth)acrylate monomer; and
ii. a crosslinking agent, and b. curing the biomaterial to form a hydrogel having a material property.

21. The method of claim 20, wherein the crosslinking agent is a photoinitiator, and the curing step comprises exposing the biomaterial to light for an exposure time so as to achieve the material property.

22. The method of claim 20, wherein the material property is an elastic modulus.

23. The method of claim 20, further comprising assembling a plurality of hydrogels to form a structure.

24. The method of claim 20, wherein the shaping device is a mold.

25. The method of claim 20, wherein the shaping device is a microfluidic channel.

26. The method of claim 20, wherein the shaping device is a 3D printing platform.

27. A kit for use in tissue engineering, comprising a biomaterial in a container, said biomaterial comprising:
a. a demineralized, decellularized bone extracellular matrix material (bECM) functionalized with a crosslinkable (meth)acrylate monomer; and
b. a crosslinking agent.

28. The kit of claim 27, further comprising instructions for delivering an amount of the biomaterial and curing the bECM to form a hydrogel.

29. The kit of claim 27, further comprising a delivery device for delivering an amount of the biomaterial.

30. The kit of claim 29, wherein the delivery device is configured to be operably connected to the container for delivering the biomaterial directly from the container.

31. The kit of claim 27, wherein the crosslinking agent is a photoinitiator.

32. The kit of claim 27, wherein the biomaterial is formulated as a paste.

33. The kit of claim 27, wherein the biomaterial is a lyophilized powder.

34. A method of making a biomaterial having a tunable material property, comprising the steps:
a. demineralizing powdered bone by treating the powdered bone with acid to produce demineralized bone material;
b. extracting lipids from the demineralized bone material;
c. decellularizing the demineralized bone material to produce demineralized bone extracellular matrix material;
d. solubilizing collagen in the demineralized bone extracellular matrix material; and
e. reacting the demineralized bone extracellular matrix material with a (meth)acrylic reagent to produce a crosslinkable bone extracellular matrix material, wherein said reacting is performed at a pH of from about 8 to about 10.

35. The method of claim 34, further comprising:
a. centrifuging a solution produced by any one of steps a. through d. to produce a precipitate and a supernatant;
b. filtering solids of a size from the supernatant; and
c. performing the subsequent step on the solids.

36. The method of claim 34, wherein the (meth)acrylic reagent is methacrylic anhydride.

37. The method of claim 34, wherein the reacting step is performed with an amount of (meth)acrylic reagent of from about 0.1 ml to about 0.3 ml per gram of demineralized bone extracellular matrix material.

38. The method of claim 34, wherein the reacting step is performed at a reaction temperature of from about 30° C. to about 55° C.

39. The method of claim 34, wherein the reacting step is performed for a reaction time of from about 1 to about 5 hours.
